(19) United States
(12) Patent Application Publication
(10) Pub. No.: US 2026/0137420 A1
(43) Pub. Date: May 21, 2026

(54) SURGICAL KNIFE WITH LASER-IMPRINTED MEASUREMENTS

(71) Applicant: Jayme Alencar De Oliveira Filho, Virgina Beach, VA (US)

(72) Inventor: Jayme Alencar De Oliveira Filho, Virgina Beach, VA (US)

(21) Appl. No.: 18/949,517

(22) Filed: Nov. 15, 2024

(51) Int. Cl. A61B 17/3213; A61B 90/00

(52) U.S. Cl. CPC A61B 17/3213; A61B 2090/062; A61B 2560/04

(57) ABSTRACT

A surgical knife including indices corresponding to a measurement is disclosed, including a handle portion releasably connected to a blade. The blade includes a tip, a cutting edge, and a body portion. A plurality of indices are provided on a top of the body portion to enable the accurate measurement of a depth while utilizing the blade to make an incision.

### TECHNICAL FIELD

The embodiments disclosed herein generally relate to a surgical knife used as a medical instrument.

### BACKGROUND

A surgical knife (otherwise referred to as a scalpel) is a sharp bladed instrument used for surgery and dissection and are constructed using various metals such as hardened and tempered steel, stainless steel, titanium, etc. Their design aids the surgeon or other user in executing precise cuts. Surgical knives often consist of a handle and a blade which may be fixed or removable from the handle. In most cases, the handle is reusable while the blade is removable such that it can be replaced following use. This also allows the surgeon to select an optimal blade configuration for each particular cut or incision being made.

When making an incision or cut, it is imperative that the surgeon be cognizant of the depth at which they are cutting through the tissue to preserve as much living tissue as possible and ensure the depth is minimized to only what is required by the particular procedure. In such, the surgeon must have sufficient knowledge and experience to ensure the incision is only as deep as what is needed. To provide a more accurate determination of the depth of the incision or cut being made probes and rulers are commonly used. However, these measuring devices occupy the surgeon's hands and increase the time needed to perform the procedure. Further, utilizing another tool near the surgical site increases the risk of contamination and subsequent infection.

### SUMMARY OF THE INVENTION

This summary is provided to introduce a variety of concepts in a simplified form that is further disclosed in the detailed description of the embodiments. This summary is not intended for determining the scope of the claimed subject matter.

A surgical knife including indices corresponding to a measurement is disclosed, including a handle portion releasably connected to a blade. The blade includes a tip, a cutting edge, and a body portion. A plurality of indices are provided on a top of the body portion to enable the accurate measurement of a depth while utilizing the blade to make an incision.

The surgical knife includes color-coded indices to enable the user (e.g., a surgeon or medical professional) to quickly and efficiently identify the depth of the blade throughout the process of making an incision. This eliminates the need for additional measuring tools which are commonly used during the medical procedure to measure the incision or other aspects of the patient's anatomy, surgical flap, etc.

The indices can be applied to various sizes, shapes, and configurations of blades which are commonly used during medical procedures, allowing the surgeon to select a suitable blade which is advantageous for the task being performed.

In some aspects, the plurality of indices are laser-etched onto a top portion of the blade.

In one aspects, the plurality of indices are color-coded to correspond to one or more measurement intervals.

In some aspects, the one or more measurement intervals include a first interval corresponding to a first color. Further, the one or more measurement intervals include a second interval corresponding to a second color. For example, the first color may be red, while the second color may be yellow which allows the user to easily identify measurement intervals on the blade.

In some aspects, the first interval is 1 millimeter.

In some aspects, the second interval is 3 millimeters.

In some aspects, the blade is constructed of stainless steel.

In some aspects, the handle is constructed of one of: stainless steel or aluminum.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of the surgical knife with laser imprinted measurements, according to some embodiments;

FIG. 2 illustrates a top plan view of the surgical knife with laser imprinted measurements, according to some embodiments;

FIG. 3 illustrates a side elevation view of the surgical knife with laser imprinted measurements, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relates to a surgical knife with laser-imprinted measurements to eliminate the need to use additional instruments such as a dental ruler or dental probe when measuring surgical sites. The surgical knife enables the user to easily measure the depth of a cut or size of a surgical flap during the cutting procedure.

The surgical knife can be provided having various blade shapes and include laser-imprinted measurements in various increments such as 1 mm, 3 mm, etc. The measurements begin at the top or edge of the blade and may begin, for example, with 1 mm measurement increments and progress to 12 mm measurement increments. Measures indices are laser imprinted onto the non-cutting surface of the blade. Color coding may be used to indicate various measurement increments, allowing the user to easily identify the depth of the cut and/or size of a surgical flap.

In some embodiments, the blade may be removable from the handle such that it may be replaced as needed.

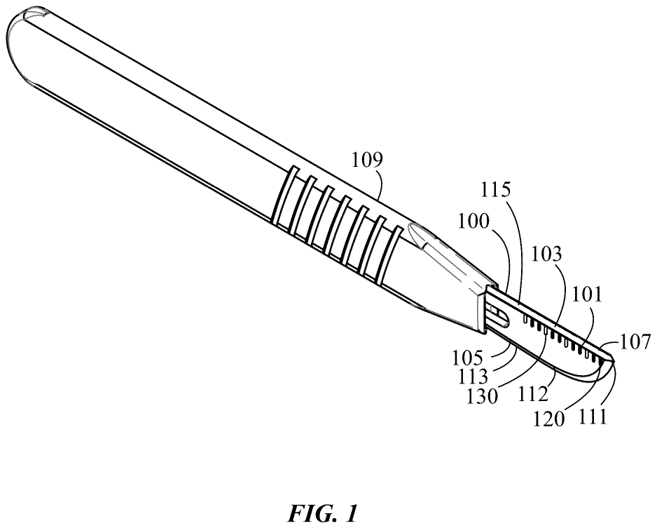

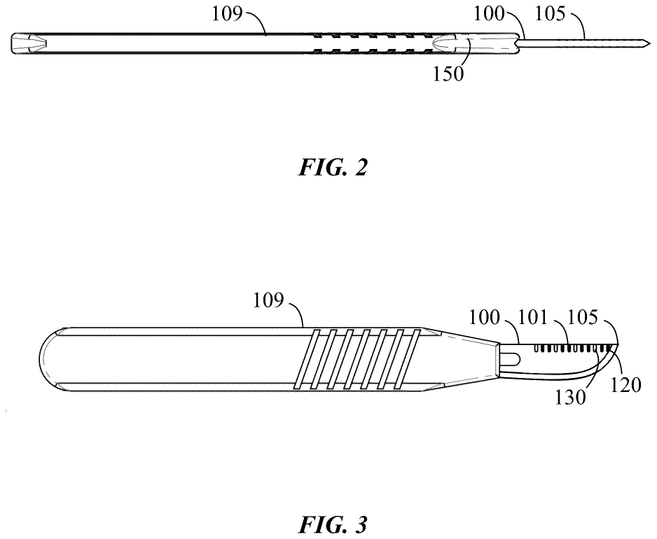

FIGS. 1-3 illustrate the surgical knife 100 having a plurality of laser-imprinted measurement indices 101 provided on the non-cutting surface 103 of the blade 105. The non-cutting surface 103 may be defined as the top 107 of the blade 105 which is positioned above the cutting edge 107. The surgical knife 100 includes a handle portion 109 releasably connected to the blade 105. The blade 105 includes a tip 111, cutting edge 112, and body portion 113. The indices 101 are provided on the top 107 of the body portion 113 to enable the accurate measurement of a depth while utilizing the blade 105 to make an incision during a medical procedure.

In some embodiments, each indices 101 may be laser-etched onto the top portion 115 of the blade 105. The indices 101 may be color-coded to correspond to one or more measurement intervals (e.g., 1 mm, 3 mm, etc.).

In some embodiments, a first interval 120 may be 1 mm intervals corresponding to 1 mm interval measurements of the indices 101. The first interval 120 markings may be identified by a first color imprinted or otherwise displayed on the first interval 120 indices 101.

In some embodiments, a second interval 130 may be 3 mm intervals corresponding to 3 mm interval measurements of the indices 101. The first interval 130 markings may be identified by a second color imprinted or otherwise displayed on the first interval 130 indices 101.

In some embodiments, the handle portion 109 may be constructed of stainless steel, plastic, metals, metal alloys, or similar materials commonly used in the construction of surgical knives. The handle portion 109 may include a blade release 150 which allows the surgeon to disconnect and replace the blade 105. This enables the surgeon to select a blade 105 having a suitable size, shape, and configuration which is best-suited for the particular task being performed during the medical procedure.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A surgical knife including indices corresponding to a measurement, comprising:
a handle portion releasably connected to a blade, the blade including a tip, a cutting edge, and a body portion; and
a plurality of indices provided on a top of the body portion, the plurality of indices to enable the accurate measurement of a depth while utilizing the blade to make an incision.

2. The surgical knife of claim 1, wherein the plurality of indices are laser-etched onto a top portion of the blade.

3. The surgical knife of claim 2, wherein the plurality of indices are color-coded to correspond to one or more measurement intervals.

4. The surgical knife of claim 3, wherein the one or more measurement intervals include a first interval corresponding to a first color.

5. The surgical knife of claim 4, wherein the one or more measurement intervals include a second interval corresponding to a second color.

6. The surgical knife of claim 5, wherein the first interval is 1 millimeter.

7. The surgical knife of claim 6, wherein the second interval is 3 millimeters.

8. The surgical knife of claim 7, wherein the blade is constructed of stainless steel.

9. The surgical knife of claim 8, wherein the handle is constructed of one of the following: stainless steel or aluminum.

10. A surgical knife including laser-etched indices corresponding to a measurement, comprising:
a handle portion releasably connected to a blade, the blade including a tip, a cutting edge, and a body portion; and
a first set of indices corresponding to a first interval and a second set of indices corresponding to a second interval, wherein each of the first set of indices and the second set of indices are laser-etched on a top portion of the body portion to enable a surgeon to assess a depth of an incision while actively utilizing the blade to make the incision.

11. The knife blade of claim 10, wherein the plurality of indices are stamped onto the top portion of the blade.

12. The surgical knife of claim 11, wherein the first set of indices and the second set of indices are color-coded to correspond to one or more measurement intervals.

13. The surgical knife of claim 12, wherein the first interval corresponds to a first color.

14. The surgical knife of claim 13, wherein the second interval corresponds to a second color.

15. The surgical knife of claim 14, wherein the first interval is 1 millimeter, and the second interval is 3 millimeters.

16. The surgical knife of claim 15, wherein the optimal blade shape, size, and configuration is selectable via the surgeon to enable the efficient and repeated replacement of the blade during a surgical procedure.

17. The surgical blade of claim 16, wherein the blade is constructed of stainless steel.

18. The surgical knife of claim 17, wherein the handle is constructed of one of the following: stainless steel or aluminum.

19. A surgical knife including laser-etched indices corresponding to a measurement, comprising:
a handle portion including a front end and a rear end, the front end including an opening to partially receive a heal of a blade, the blade including a tip, a cutting edge, the heal, and a body portion; and
a receiver positioned on the heel to releasably attach blade to the handle;
a first set of indices corresponding to a first interval and a second set of indices corresponding to a second interval, wherein each of the first set of indices and the second set of indices are laser-etched on a top portion of the body portion to enable a surgeon to assess a depth of an incision while actively utilizing the blade to make the incision.
